(19) United States
(12) Patent Application Publication
(10) Pub. No.: US 2022/0313097 A1
(43) Pub. Date: Oct. 6, 2022

(54) NON-INVASIVE PULSATILE DEVICE FOR CIRCULATORY ASSISTANCE

(71) Applicant: CARDIO INNOVATIVE SYSTEMS, Paris (FR)

(72) Inventors: Renan LE BLE, SAINT-PIERRE-DU-VAUVRAY (FR); Michel DIXMIER, PARIS (FR); Pierre CHASTANIER, PARIS (FR); Olivier BAILLIART, PARIS (FR)

(21) Appl. No.: 17/596,225

(22) PCT Filed: Jun. 11, 2019
(86) PCT No.: PCT/FR2019/051401

§ 371 Date: Dec. 6, 2021

(51) Int. Cl. A61B 5/0205; A61H 9/00

(52) U.S. Cl. CPC A61B 5/0205; A61H 9/0007; A61H 2201/165; A61H 2205/022; A61H 2205/10; A61H 2205/08; A61H 2205/06

(57) ABSTRACT

The non-invasive pulsatile circulatory support device intended to promote circulation of a blood volume in at least a part (Z) of a subject's body, the device including a flexible structure, arranged so as to be applied on at least the part of the subject's body, including a series of paired adjacent pouches extending along one another; and means for generating a physiologically synchronised pulsation fluidically connected to the flexible structure in a leak-tight manner and arranged so as to create pulsation waves in the series of pouches between the inner and outer layers, one of the paired adjacent pouches at least partially covering the other of the paired adjacent pouches (scaled structure).

### TECHNICAL FIELD OF THE INVENTION

The invention relates to a non-invasive pulsatile circulatory support device.

### STATE OF THE RELATED ART

The circulatory system forms a pressurised closed hydraulic circuit, lined internally by endothelial cells. This endothelium is continually and in a pulsatile fashion subjected to tangential shear forces essential for maintaining the physiological function thereof: vascular tonicity thanks to nitrogen monoxide synthesis, blood coagulation, inflammatory response, atherosclerosis control, immune system, angiogenesis and apoptosis.

Any pathological impairment of this endothelial function induces system dysfunction with sometimes dramatic consequences.

Cardiac support systems exist, which are used to partially or completely replace cardiac activity during surgical procedures or to restore this activity when the heart has stopped or is too weak. These support systems are, for the most part, invasive systems: they require either the introduction of a tool into a subject's body, this tool being subsequently used to create pulsations, or the drawing of blood from the subject and treatment of the blood drawn in a bulky machine outside the body followed by injection of the blood into the subject's body. In any case, the current systems are costly and complex to implement as they require specialist intervention.

A non-invasive system has been described in the document WO2010/070018 as well as in the document WO2014/080016. These documents describe a non-invasive circulatory support device including a structure composed of one or more pouches connected to a console suitable for generating pulsatile waves at the level of the pouch(es) so as to promote circulation of a blood volume in at least a part of a subject's body.

In the case of a device including a single pouch, the drawback is the inability to precisely control the distribution of the pulsatile wave or to modulate same according to the pressure zones of the pouch on the part of the body of the subject to be treated.

In the case of a device including several pouches, the pouches are positioned adjacently in pairs, which makes it possible to partially resolve the previous drawback of the single pouch. However, there is a loss of continuity in the propagation of the pulsation waves generated at the level of the junction between two adjacent pouches. Furthermore, this junction induces, during the operation of the device, a tourniquet effect harmful for the subject.

Further non-invasive ECP (External Counter Pulsation) systems produce an external counterpulsation inducing blood reflux. In a known manner, these systems act as a tourniquet on the lower part of the body, which directs the blood upstream, towards the vital organs, of which the heart. In other words, ECP interrupts blood circulation due to a tourniquet effect, with a back pressure of the order of at least 130 mmHg. In some ECP systems, the tourniquet effect has become sequential, then has been synchronised with the heart rate, the tourniquet effect then being induced during the diastole. A fluid mechanics type effect is created in this case: creation of an aortic retrograde pressure wave which increases coronary perfusion; tourniquet effect, without significant increase in venous return, therefore ineffective on the right ventricular preload and on the cardiac output. This technique requires a pressure, applied to the lower part of the body, of the order of 200 to 300 mmHg, which is largely greater than the subject's physiological systolic pressure. This pressure with sequential tourniquet effect induces vessel compression, de facto triggering a mechanical pressure effect on the vessels. A harmful effect on endothelial cells and blood vessels is unfortunately encountered with this technique.

Pressotherapy devices and methods generally consisting of a venolymphatic drainage method are also known. Usually, venolymphatic drainage by means of a pressotherapy method uses physiological pressure gradient apparatuses, with inflatable boots activated by an air compressor (the pressure increases progressively, ranging from 40 to 80 millibar). The purpose of this massage is to activate lymph circulation, stimulate the immune defence system, and induce deep relaxation. Thus, the phenomenon of heavy legs and cellulite gradually lessens. However, pressotherapy has the drawback of compressing blood vessels over a long period which impedes blood circulation, outside of any cardiac rhythm. Moreover, so as to activate lymph circulation, pressotherapy requires significant compression of the lymphatic system.

### DESCRIPTION OF THE INVENTION

An aim of the invention is to provide a non-invasive pulsatile circulatory support device which enables homogeneous, continuous and regular pulsation wave propagation while enabling precise control and/or modulation of the pulsation waves according to the pressure zones of the device on the part of the subject's body.

For this purpose, the invention relates to a non-invasive pulsatile circulatory support device intended to promote circulation of a blood volume in at least a part of a subject's body, the device comprising a flexible structure, arranged so as to be applied on at least the part of the subject's body, including a series of paired adjacent pouches extending along one another, and, means for generating a pulsation fluidically connected to the flexible structure in a leak-tight manner and arranged so as to create pulsation waves in the series of pouches between the inner and outer layers, one of the paired adjacent pouches at least partially covering the other of the paired adjacent pouches.

Advantageously, but optionally, the device according to the invention has at least one of the following technical features:
the pouches of the series of pouches are mounted in a scaled structure;
each of the pouches comprises an elastic flexible inner layer on the side of the patient's body and a more rigid outer layer;
each of the pouches includes a pulsation fluid intake duct fluidically connected with means for generating a pulsation;
the flexible structure is a multilayer casing wherein the series of paired adjacent pouches forms a distribution layer;
the multilayer casing includes a generation layer covering the distribution layer and fluidically connected to the means for generating a pulsation;
the generation layer includes a series of paired adjacent inflatable pads extending along one another;
each of the inflatable pads includes a pulsation fluid intake duct fluidically connected with means for generating a pulsation;
the multilayer casing includes a non-stretch flexible outer layer;
the device includes a mask arranged so as to be disposed on at least a part of a subject's face;
the device includes pants;
the device includes a jacket;
the device includes a glove; and,
the device includes a boot or a stocking.

The invention also relates to a non-invasive pulsatile circulatory support assembly covering several parts of a subject's body comprising at least one device having a least one of the above technical features for each of the parts of the subject's body.

### BRIEF DESCRIPTION OF THE FIGURES

Further features and advantages of the invention will emerge on reading the following description of an embodiment of the invention. In the appended drawings:

FIG. 1 is a schematic representation of a non-invasive pulsatile circulatory support device according to the invention;

FIG. 2 is a schematic representation of an example of a multilayer structure forming a pouch used in the device in FIG. 1;

FIG. 3 is a schematic representation of a pulsatile mask according to the invention;

FIG. 4 is a schematic representation of pulsatile pants according to the invention; and,

FIG. 5 is a schematic representation of a second embodiment of the non-invasive pulsatile circulatory support device according to the invention.

For more clarity, identical or similar elements are identified by identical reference signs in all the figures.

### DETAILED DESCRIPTION OF AN EMBODIMENT

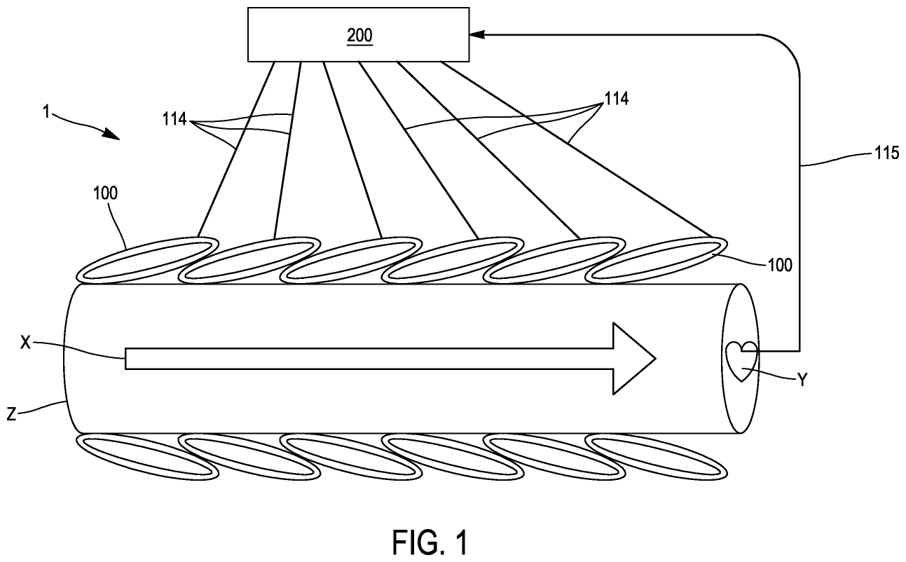

With reference to FIG. 1, we will describe a non-invasive pulsatile circulatory support device 1 according to the invention. The non-invasive pulsatile circulatory support device 1 according to the invention is intended to be positioned on a part Z of a subject's body so as to promote, during use, circulation of a blood volume in the part Z of the subject's body, in the direction X towards the subject's heart Y.

The part Z of the subject's body is represented schematically herein by a revolving cylindrical tube portion which is used herein merely by way of illustration. In particular, the part Z may be an upper or lower limb of the subject, or indeed the latter's torso or head. The structure of the non-invasive pulsatile circulatory support device 1 according to the invention enables the latter to conform to all shapes and geometries of the part Z of the subject's body to be equipped with such a non-invasive pulsatile circulatory support device 1 according to the invention.

The non-invasive pulsatile circulatory support device 1 according to the invention includes herein a series of paired adjacent pouches 100. The series of pouches 100 illustrated in FIG. 1 comprises six pouches 100 uniformly distributed along the part Z of the subject's body. The number of pouches 100 varies according to the part Z of the subject's body concerned, it may be between one and six, or indeed greater than six. Each of the pouches 100 surrounds, herein, the part Z of the subject's body. Each of the pouches 100 includes an intake duct 114 suitable for fluidically connecting the pouch 100 to a pulsatile console 200 forming means for generating a pulsation. The same pulsatile console 200 of the non-invasive pulsatile circulatory support device 1 according to the invention controls all the pouches 100 of the series of pouches 100, the different pouches 100 being independent from one another. The means for generating a pulsation 200 forming the pulsatile console may have different structures which are described in the document WO 2010/070018 to which reference may be made for further information. Therefore, they will not be described further herein.

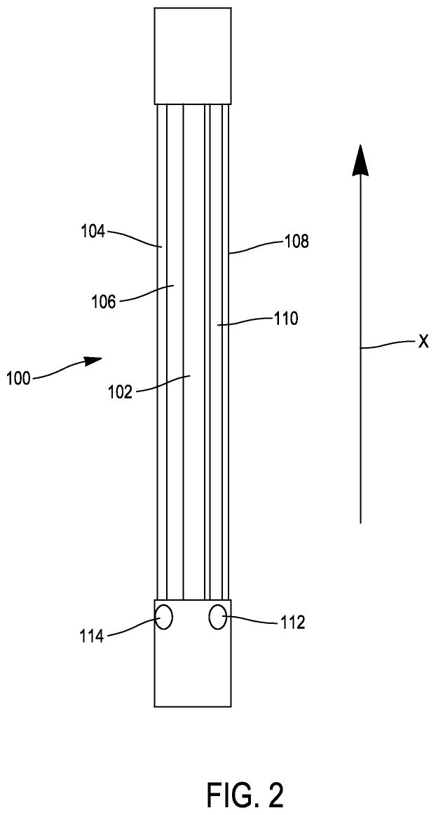

The pouches 100 are therefore inflatable via the intake duct 114 thereof. For this, they have an elastically deformable wall in a simpler embodiment. With reference to FIG. 2, we will subsequently describe a further embodiment of the pouches 100.

On the part Z of the subject's body, the paired adjacent pouches 100 of the series of pouches 100 are mounted such that one of the paired adjacent pouches at least partially covers the other of the paired adjacent pouches. This partial overlap makes it possible to obtain a continuity in the propagation of the pulsatile waves generated by the pulsatile console 200. In particular, the non-invasive pulsatile circulatory support device 1 according to the invention thus embodied enables, during operation, an uninterrupted, total, regular and homogeneous centripetal massaging action (along the direction X) in waves, while retaining the possibility of applying differentiated pressures, even none if needed, on certain zones of the part Z of the subject's body, according to needs (presence of burn, wound, swelling, etc.). Preferably, the pulsatile console 200 is arranged so as to synchronise the pulsatile waves generated thereby with a physiological signal 115 received thereby from a sensor placed on the subject. The physiological signal originates for example from the cardiac rhythm and in particular corresponds to the diastole of said cardiac rhythm. Furthermore, data on the subject's blood pressure may be observed in parallel so as to adapt the pressures generated by the console 200 to said subject. In the case of cardiac arrhythmia, the physiological signal may originate from the subject's respiratory cycle.

In a particular arrangement, the pouches 100 are mounted in a scaled structure as illustrated in FIG. 1. Further arrangements of pouches 100 may be envisaged for the non-invasive pulsatile circulatory support device 1 according to the invention, as long as the paired adjacent pouches at least partially covers the other of the of the paired adjacent pouches in the series of pouches 100 forming the non-invasive pulsatile circulatory support device 1 according to the invention.

FIG. 2 is a schematic representation of an embodiment of a multilayer structure forming each of the pouches 100 used in the non-invasive pulsatile circulatory support device 1 according to the invention.

The multilayer structure forming each of the pouches 100 represented in FIG. 1 includes:
an inner layer 102 made of an elastic material, for example of neoprene, polyurethane, latex, etc.,
an outer layer 104 formed of an inflatable pad capable of having an outer wall consisting of a rigid or non-stretch flexible material in the plane thereof guiding the propagation of the compression waves towards the inside of the body, and
an intermediate layer 106 containing a fluid, which may also be a gel, microbeads or a combination of both enabling the propagation of a progressive pulsatile pressure wave towards the heart along the natural and physiological direction of venous and lymphatic drainage in the part whereon said structure is applied. Hereinafter in the description, the natural and physiological direction of venous and lymphatic drainage is the direction X represented in FIGS. 1 and 2.

The multilayer structure forming each of the pouches 100 further includes an additional layer 108, including a cavity 110 made of biocompatible material, and including a microporous wall intended to come into contact with the subject's body. The cavity 110 may be filled with a biocompatible and/or biological fluid substance via a connector 112. The microporous wall is in direct contact with the skin of the subject's body. During the pulsations, the substance contained in the space 110 of this layer 108 is applied to the subject's body by passing through the microporous part.

The outer layer 104 is fluidically connected in a leak-tight manner with the pulsation means 200 for creating pulsation waves in the pouches 100 of the non-invasive pulsatile circulatory support device 1 according to the invention thanks to an intake duct 114.

To carry out the propagation of the pulsations along the part of the body whereon the pouch 100 is applied, the intermediate layer 106 includes a substance of variable consistency, gelatinous, granular or other, and distributing each of the pulsations progressively along said multilayer structure forming each of the pouches 100 in the direction X.

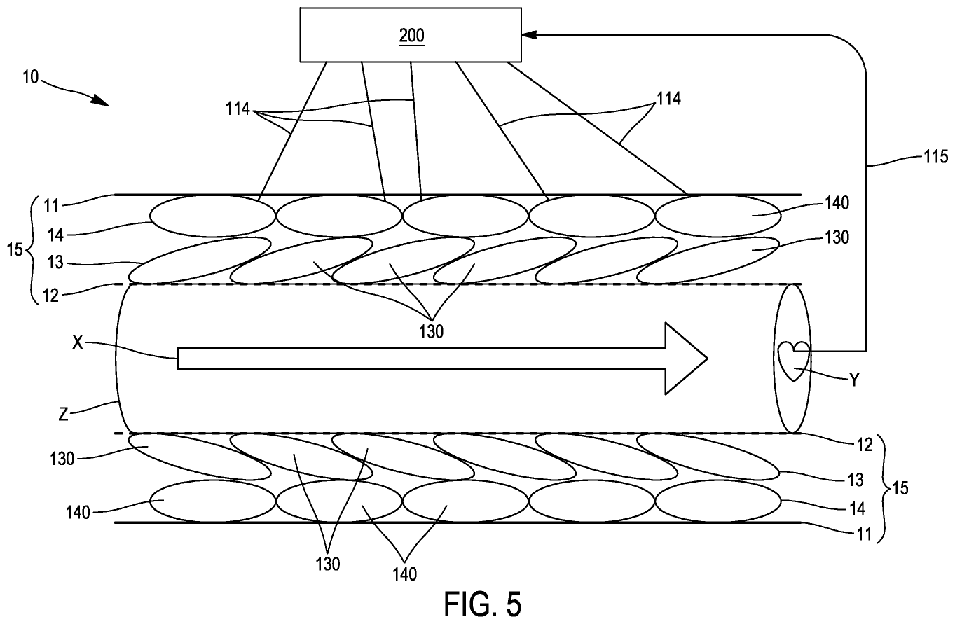

With reference to FIG. 5, we will now describe a second embodiment of a non-invasive pulsatile circulatory support device 10 according to the invention.

The non-invasive pulsatile circulatory support device 10 according to the invention includes a multilayer casing 15 which surrounds the part Z of the subject's body when fitting the non-invasive pulsatile circulatory support device 10 according to the invention. The casing 15 includes:
An inner layer 12 which is formed of an elastic material, for example of neoprene, polyurethane, latex, etc.;
A distribution layer 13 including herein a series of paired adjacent pouches 130;
A generation layer 14 including herein a series of paired adjacent inflatable pads 140; and,
An outer layer 11 made of a non-stretch flexible material in the plane thereof or of a rigid material.

The inner layer 12 is presented in the general form of a film intended to come into contact with the part Z of the subject's body and suitable for deforming and stretching during the use of the non-invasive pulsatile circulatory support device 10 according to the invention. According to an alternative embodiment, the inner layer 12 is microporous and may be impregnated with a biocompatible and/or biological fluid substance. During the pulsations, the substance impregnating the micropores of the inner layer 12 is applied to the subject's body.

The series of pouches 130, forming the distribution layer 13 and illustrated in FIG. 5, comprises, herein, six pouches 130 uniformly distributed along the part Z of the subject's body. Each of the pouches 130 surrounds, herein, the part Z of the subject's body and is separated from said part Z of the subject's body by the inner layer 12 then serving as an interface between each of the pouches 130 and the part Z of the subject's body, the different pouches 130 being independent from one another. The pouches 130 have an elastically deformable wall and are filled with an incompressible fluid enabling the propagation of a progressive pulsatile pressure wave towards the heart along the natural and physiological direction of venous and lymphatic drainage in the part Z whereon the non-invasive pulsatile circulatory support device 10 according to the invention is applied. For example, this incompressible fluid is a gelatinous, granular or other fluid. As for the first embodiment illustrated in FIG. 1 and described above, the paired adjacent pouches 130 of the series of pouches 130 are mounted such that one of the paired adjacent pouches at least partially covers the other of the paired adjacent pouches. This partial overlap makes it possible to obtain a continuity in the propagation of the pulsatile waves generated by the pulsatile console 200, while retaining the possibility of applying differentiated pressures, even none if needed, on certain zones of the part Z of the subject's body, according to needs (presence of burn, wound, swelling, etc.).

The series of pads 140, forming the generation layer 14 and illustrated in FIG. 5, includes, herein six pads 140 uniformly distributed along the part Z of the subject's body. Each of the pads 140 surrounds, herein the distribution layer 13 such that the latter is sandwiched between the inner layer 12 and said generation layer 14. Thus, the generation layer 14 covers the distribution layer 13. Furthermore, each of the pads 140 includes an intake duct 114 suitable for fluidically connecting the pad 140 to the pulsatile console 200 forming means for generating a pulsation. The same pulsatile console 200 of the non-invasive pulsatile circulatory support device 10 according to the invention controls all of the pads 140 of the series of pads 140, the various pads 140 being independent from one another. The pads 140 are therefore inflatable via the intake duct 114 thereof. For this, they have an elastically deformable wall. The fluid used in the pads 140 is air, any other fluid being alternatively usable. Thus, the non-invasive pulsatile circulatory support device 10 according to the invention thus embodied enables, during operation, an uninterrupted, total, regular and homogeneous centripetal massaging action (along the direction X) in waves, while retaining the possibility of applying differentiated pressures, even none if needed, on certain zones of the part Z of the subject's body, according to needs (presence of burn, wound, swelling, etc.). The pressures are then transmitted from the generation layer 14 towards the part Z of the subject's body by the inner layer 12.

The outer layer 11 is presented in the form of a flexible film, as for the inner layer 12. The outer layer 11 is non-stretch in the plane thereof such that the deformations of the series of inflatable pads 140 generated by the console 200 in the generation layer 14 can only be carried out essentially in the direction of the part Z of the subject's body, so as to produce the pressure waves to be applied thereto via the distribution layer 13. However, the outer layer 11 is sufficiently flexible to enable the fitting of the non-invasive pulsatile circulatory support device 10 according to the invention around the part Z of the subject's body.

In an alternative embodiment, the non-invasive pulsatile circulatory support device 10 according to the invention includes means for regulating a temperature inside a multilayer casing 15, and in particular in the vicinity of the inner layer 12.

We will now describe different pulsatile elements according to the invention.

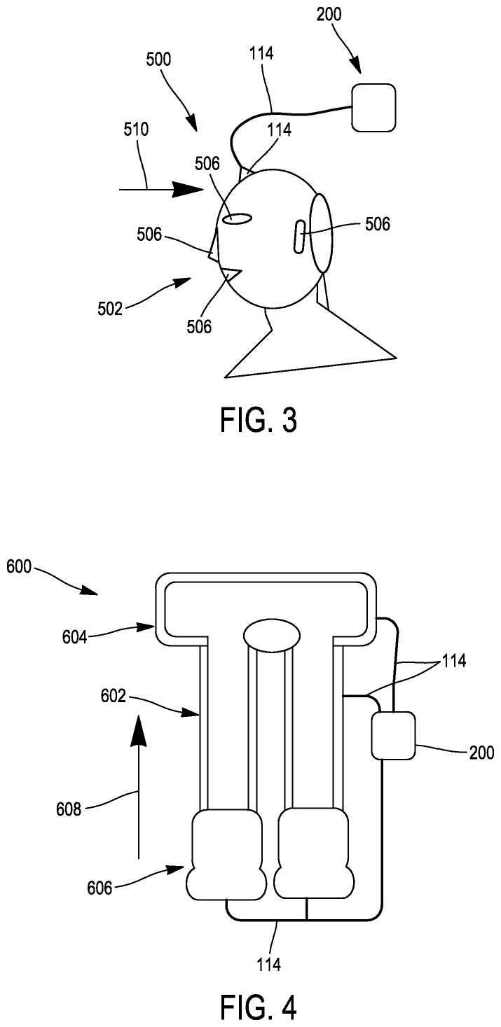

FIG. 3 is a schematic representation of a pulsatile mask 500 according to the invention. The pulsatile mask 500 is composed of a facial part 502 embodied with a non-invasive pulsatile circulatory support device 1 according to the invention as described with reference to FIGS. 1, 2 and 5.

The facial part 502 may have openings 506, at the levels of the eye sockets, mouth, nose and ears. A set of intake ducts 114 connects the pulsatile console 200 to the set of pouches 100 of the facial part 502. Each of the pulsations generated and controlled via the set of pouches 100 is propagated progressively in the facial part 502 so as to perform a facial massage. A horizontal axis conveyed by the arrow 510 represents the path of the pulsatile waves towards the cavernous circuit.

The mask 500 serves as non-invasive pulsatile circulatory support to treat venolymphatic stasis of the face and neck. It is worn applied to the face and in part to the scalp. The facial part 502 may operate in rhythmic and regular synchronisation and in harmony with the subject's cardiorespiratory rhythms.

The mask 500 further has the following functions:
Primary: restoration and repair of side-effects of endothelial dysfunction by the application of shear forces synchronised with the diastole, reducing lymphatic and venous congestion; and
Secondary: haemodynamic improvement of blood circulation.
improvement of skin circulation, accelerating the uptake and penetration of existing cosmetic products such as anti-ageing or skin care products.

The inner layer of the pouches 100 of the mask 502 may be modelled on a biological or biocompatible material mask, adapted to the shape of the subject's face and neck. The internal surface may be microporous enabling the diffusion towards the skin of fluids of cosmetic nature with or without temperature variation of the products or fluids used, according to the indications.

FIG. 4 is a schematic representation of pulsatile pants 600 according to the invention.

The pulsatile pants 600 composed of a leg part 602, a waistband part 604, and optionally a boot part 606. Each of the parts cited above forms a non-invasive pulsatile circulatory support device 1 according to the invention. In this version of the invention, the pants 600 contain no microporous layer.

The pulsatile waves start at the level of the boot part 606 from the pulsatile console 200 via sets of intake ducts 114 fluidically connecting the different pouches 100 to said pulsatile console 200. Each of the pulsations is then propagated towards the heart along an axis conveyed by the arrow 608.

This system, besides the functions described above for the pulsatile mask 500, has a both repairing and preventive use:
repairing endothelial dysfunction thanks to the shear forces by promoting angiogenesis; and
preventive against endothelial dysfunction.

In a particular version, the pulsatile pants 600 may comprise a first layer in contact with the skin through personal clothing.

Modifications of the rear part of the waistband 604 may be envisaged for massaging the lower lumbar spine.

Communications between the different parts (pants, waistband, legs) are coordinated and synchronised with the diastole.

Similarly, a pulsatile sleeve, a pulsatile jacket, pulsatile underwear, pulsatile boots, pulsatile gloves as well as a full pulsatile suit formed using one or more non-invasive pulsatile circulatory support devices 1 according to the invention may be envisaged.

A full pulsatile suit may also be obtained by assembling a mask, a jacket, pulsatile pants, pulsatile gloves and pulsatile shoes. In this case, according to a first embodiment, each pulsatile assembly may be associated with dedicated pulsation means 200. According to a second embodiment, single pulsation means 200 may be used for all the pulsatile assemblies forming the pulsatile suit.

The propagation of the pulsatile pulses is synchronised on the basis of several distal sources such as the pulsatile boots or pulsatile gloves.

Each pulsatile assembly may be used separately according to the subject's needs.

Each pulsatile device according to the invention is a non-invasive circulatory support device, enabling a progressive reduction of the stagnant venolymphatic capacitance. By increasing the right cardiac preload through an increase in the venolymphatic return, the device according to the invention improves cardiac function. In parallel, through an endothelial stimulation effect and through a vasodilation effect, the device according to the invention, due to the use thereof, induces overall haemodynamic improvement. In the long term, the shear forces produced by the pulsatile device according to the invention will restore and preserve endothelial function. This more physiological method, capable of reducing morbidity and mortality, is applicable to both children and adults as well as to animal subjects. In addition to the shear forces produced, the pulsatile device according to the invention generates a draining effect on the part Z of the subject's body, which improves the blood microcirculation and therefore venous endothelium stimulation further.

Obviously, numerous modifications may be made to the invention without leaving the scope thereof.

1. Non-invasive pulsatile circulatory support device (1;10;500;600) intended to promote circulation of a blood volume in at least a part (Z) of a subject's body, the device comprising:
a flexible structure (15), arranged so as to be applied on at least the part of the subject's body, including:
i. a distribution layer (13) comprising a first series of paired adjacent pouches (130) extending along one another, one of the paired adjacent pouches at least partially covering the other of the paired adjacent pouches;
ii. a generation layer (14), covering the distribution layer (13), comprising a second series of paired adjacent pouches (140) extending along one another; and,
means (200) for generating a pulsation fluidically connected to the flexible structure in a leak-tight manner and arranged so as to create pulsation waves in the second series of pouches of the generation layer between the inner and outer layers,
wherein the second series of pouches (140) of the generation layer includes a series of paired adjacent inflatable pads (140) extending along one another, the first series of paired pouches (130) of the repartition layer including pouches filled with an incompressible fluid enabling the propagation of the pulsation waves created within the generation layer by the means (200) for generating.

2. The device according to claim 1, wherein the pouches of the second series of pouches are mounted in a scaled structure.

3. The device according to claim 1, wherein each of the pouches of the second series of pouches (140) comprises an elastic flexible inner layer (102) on the side of the patient's body and a more rigid outer layer (104).

4. (canceled)

5. (canceled)

6. (canceled)

7. (canceled)

8. The device according to claim 1, wherein each of the inflatable pads includes a pulsation fluid intake duct (114) fluidically connected with means for generating a pulsation.

9. The device according to claim 1, wherein the flexible structure (15) includes a non-stretch flexible outer layer (11).

10. The device according to claim 1, further including a mask (500) arranged so as to be disposed on at least a part of a subject's face.

11. The device according to claim 1, further including pants (600).

12. The device according to claim 1, further including a jacket.

13. The device according to claim 1, further including a glove.

14. The device according to claim 1, further including a boot (606) or a stocking.

15. Non-invasive pulsatile circulatory support assembly covering several parts of said subject's body, comprising at least two devices selected from the list of a mask, pants, jacket, glove, boot and stocking.
